(19) United States
(12) Patent Application Publication
(10) Pub. No.: US 2013/0101635 A1
(43) Pub. Date: Apr. 25, 2013

(54) METHOD OF PREPARING SELF-ASSEMBLED EXTRACELLULAR MATRICES AND USE OF SELF-ASSEMBLED EXTRACELLULAR MATRICES FORMED BY USING THE METHOD

(71) Applicants: Kwideok PARK, Seoul (KR); Soon Eon BAE, Seoul (KR); Dong Keun HAN, Seoul (KR)

(72) Inventors: Kwideok PARK, Seoul (KR); Soon Eon BAE, Seoul (KR); Dong Keun HAN, Seoul (KR)

(73) Assignee: Korea Institute of Science and Technology, Seoul (KR)

(21) Appl. No.: 13/650,670

(22) Filed: Oct. 12, 2012

(30) Foreign Application Priority Data
Oct. 14, 2011 (KR) 10-2011-0105545

(51) Int. Cl. C12N 5/00; A61K 35/32; A61K 35/28

(52) U.S. Cl. CPC C12N 5/0062; A61K 35/28; A61K 35/32
USPC 424/400; 435/395; 435/375; 435/377; 435/325; 424/93.7

(57) ABSTRACT

Methods of preparing a self-assembled matrix from cell-derived extracellular matrice, and of inducing cell proliferation and differentiation by using the self-assembled matrix, and applying the self-assembled matrix into cell therapy. In detail, unique extracellular matrices obtained from particular cells are collected and then subjected to decellularization in order to form a self-assembled matrix including nano fibers only formed of extracellular matrix. In addition, the matrix prepared as described above provides a platform that is effective for the induction of the mass proliferation or differentiation of cells, and thus, may be applied in manufacturing cell therapy products.

### CROSS-REFERENCE TO RELATED PATENT APPLICATION

This application claims the benefit of Korean Patent Application No. 10-2011-0105545, filed on Oct. 14, 2011, in the Korean Intellectual Property Office, the disclosure of which is incorporated herein in its entirety by reference.

### BACKGROUND OF THE INVENTION

1. Field of the Invention

The present invention relates to a method of preparing a self-assembled matrix using an extracellular matrix, use of the self-assembled matrix in cell proliferation and differentiation, and use of the self-assembled matrix in treating cells.

2. Description of the Related Art

Technology related to fundamental and applied tissue engineering has been advanced for the purpose of developing transplantable artificial tissues as part of regenerative medicine. Specifically, studies for stem cell proliferation and differentiation, development of cytocompatible and biocompatible three-dimensional scaffolds, and construction of a variety of tissue engineering tools are now the most active research areas in regenerative medicine. Regarding stem cells, technology for mass proliferation of undifferentiated stem cells, and technology for inducing differentiation of stem cells into particular cells are important. In addition, two or three-dimensional scaffolds that are used to deliver stem cells or tissue cells therein are critical for the development of artificial tissues and organs.

Typically, scaffolds are prepared from synthetic or natural polymers or their composites, and are manufactured into three-dimensional structures which have a variety of morphologies and properties. Examples of synthetic biodegradable polymers are polyglycolic acid (PGA), polylactic acid (PLA), poly(lactic acid-co-glycolic acid) (PLGA), poly-ε-caprolactone (PCL), and derivatives and copolymers thereof. Examples of natural polymers are collagen, alginate, hyaluronic acid, gelatin, chitosan, fibrin, silk, and derivatives thereof.

The most critical one to solve these technical barriers is the development of a core technology with regard to the scaffold that can generate a cytocompatible surface environment within or onto the scaffold. Since a major role of scaffolds is to provide an environment for cell transplantation and the improvement of survival rates of transplanted cells, properties of the scaffold surface to which cells attaches plays a critical role in determining cell behaviors. The scaffold surface to which cells directly attach is a critical factor in scaffold design, and in general, cell adhesion to natural polymers is much easier to accomplish than that of synthetic polymer.

In consideration of such characteristics, among extracellular matrix components that are recognized by cell surface receptors, fibronectin, bitronectin, laminin, or an arginine-glycine-aspartic acid (RGD) peptide can be coated or grafted on a scaffold (Bhati et al., 2001, Lin et al., 2009). In addition, the scaffold surface can be treated with a natural polymer, such as collagen, gelatin, fibrin, etc. (Mcbane et al., 2011). In addition, a hybrid scaffold can be prepared by mixing synthetic and natural polymers at a fixed ratio (Ekaputra et al., 2011). However, these approaches are only partly effective in ensuring cell adhesion and especially stem cell differentiation. In fact, current technologies of surface modifications have limitations in creating a biomimetic surface environment that the cells recognize as being natural and autogenous.

Accordingly, current cell transplantation performed by scaffold surface adhesion has many limitations in generating whole tissue regeneration. In particular, regarding stem cells, there are many technical difficulties in controlling behaviors of stem cells, such as multipotent loss or un-intended differentiation, in the procedure of cell adhesion and proliferation on a conventional plastic culturing plate or a natural polymer-coated surface. Recent research results show that in addition to chemical signals, such as a growth factor, physical factors, such as a surface structure or mechanical elements, play an important role in determining differentiation orientation of stem cells (Engler et al., 2006).

To solve these problems, the present invention provides a two-dimensional matrix developed by using extracellular matrix components secreted from a particular cell for a predetermined period of time after the cell attaches to the surface by using a cell friendly method compared to a conventional coating method. The matrix may vary according to cells, and when cells were actually cultured, excellent proliferation was obtained and in the case of stem cells, the matrix provided a microenvironment advantageous for differentiation into a particular cell. In addition, according to the present invention, proliferation and differentiation of cells may be achieved on the matrix's unique the natural microenvironment (components and structure) of the matrix, leading to high possibility for the development of cell therapy products prepared by combining the matrix and cells

### SUMMARY OF THE INVENTION

The present invention provides a method of preparing a self-assembled matrix that consists of extracellular matrices, the method including culturing cells on a 2-dimensional surface to prepare an extracellular matrix and decellularizing the 2-dimensional surface on which cells are cultured.

The present invention also provides a self-assembled matrix that consists of extracellular matrices.

The present invention also provides an in-vitro evaluation method of identifying effects of a self-assembled matrix that consists of extracellular matrices on cell adhesion, proliferation or differentiation.

The present invention also provides use of tissue cells or stem cells that are proliferated in great quantities through sub-culturing on a matrix microenvironment that is effective for cell adhesion and proliferation in cell therapy for tissue regeneration or disease treatment.

### BRIEF DESCRIPTION OF THE DRAWINGS

The above and other features and advantages of the present invention will become more apparent by describing in detail exemplary embodiments thereof with reference to the attached drawings in which:

FIG. 1 shows a scanning electron microscope (SEM) image of a self-assembled extracellular matrix obtained by decellularization (A: FDM, B: PDM, and C: CHDM), and

the image of FIG. 1 shows the surface of a self-assembled matrix formed by decellularization in which nano-sized fibers are tangled to form a network and each of the matrices has a distinguishing surface structure;

FIG. 2 shows assay results of self-assembled matrix obtained by immunofluorescent staining (A: FDM, B: PDM, and C: CHDM), and

to assay protein components that constitute self-assembled matrices, information about the respective matrices (FDM, PDM, and CHDM) was able to be obtained by immunofluorescent staining of representative components, such as fibronectin, collagen, or laminin;

FIG. 3 shows CCK-8 assay results of proliferation of bone-derived stem cells on a self-assembled matrix (A: FDM, B: PDM, and C: CHDM), and it was confirmed that cell proliferation on the matrix was better than that of a control;

FIG. 4 shows differentiation effects of bone-derived stem cells (BMSC) on a self-assembled matrix (histological staining: Alizarin red staining) (A: FDM, B: PDM, and C: CHDM), osteoblast differentiation of bone-derived stem cells was induced on the respective self-assembled matrices, and then, calcium formation and distribution were confirmed by histological staining, and results obtained thereby show that bone differentiation was much promoted on a particular matrix (FDM); and

FIG. 5 shows images of chondrocyte pellets prepared by mass proliferation of chondrocyte isolated from a cartilage of rat obtained by sub-culturing (P4) on a self-assembled matrix or a typical culturing plate. The chondrocyte was cultured for 4 weeks. Results obtained thereby show that in terms of pellet size, cartilage specific staining, total glycosaminoglycan (GAG) quantity, chondrocyte that is proliferated on a self-assembled matrix had a higher cartilage tissue generative ability than that on the typical culturing plate (PGCP: plate-grown chondrocyte pellet, MGCP: matrix-grown chondrocyte pellet).

### DETAILED DESCRIPTION OF THE INVENTION

Expressions such as “at least one of,” when preceding a list of elements, modify the entire list of elements and do not modify the individual elements of the list.

According to an aspect of the present invention, a method of preparing a self-assembled matrix that consists of extracellular matrices includes culturing cells on a 2-dimensional surface to form extracellular matrices and decellularizing the 2-dimensional surface on which the cells are cultured.

The method of preparing the self-assembled matrix will now be described in detail.

The method may include culturing cells on a 2-dimensional surface to form extracellular matrices.

As used herein, the term “extracellular matrix derived from cells” refers to an extracellular part that is generated from a material secreted from cells when the cells are cultured for a predetermined period of time. The cells may be animal cells. The cells may be tissue cells. The term “self-assembled matrix that consists of extracellular matrices” refers to a thin film of an extracellular matrix secreted from cells after nuclei thereof are removed from cultured cells.

The obtained extracellular matrix may have a predetermined pattern of nano or micro-sized fiber bundle.

An extracellular matrix contains a variety of proteins required for cell adhesion and cell transduction, and also a growth factor that is necessary for differentiation of stem cells and cytokine. Also, matrix components that are selected from cells and have a nano fiber shape form a self-assembled nanostructure, thereby forming a natural biomedical microenvironment.

In addition, since extracellular matrix components and a self-assembled structure may vary according to cells, various biominetic structures may be formed. Accordingly, the cell-derived self-assembled matrix is effective for cell adhesion and cell proliferation, and in particular, as a physical signal, may largely affect a differentiation of stem cells into a particular cell.

Regarding the culturing of the method, a material for forming the surface for culturing cells may be any one selected from the group consisting of a biodegradable organic polymer, inorganic components, a natural polymer, and a metallic material.

The biodegradable organic polymer may be any one selected from the group consisting of polyglycolic acid (PGA), polylactic acid (PLA), poly(lactic-co-glycolic acid) (PLGA), poly-ε-caprolactone (PCL), polyamino acid, derivatives, and copolymers thereof.

The inorganic components may be any one selected from the group consisting of hydroxyapatite (HA), biphasic calcium phosphate, tricalcium phosphate (TCP), calcium carbonate, and glass ceramics.

The natural polymer may be any one selected from the group consisting of collagen, alginate, hyaluronic acid, gelatin, chitosan, fibrin, and silk.

The metallic material may be any one selected from the group consisting of titanium, stainless steel, cobalt-chromium, magnesium, and alloys thereof

Regarding the culturing of the method, cells cultured on the 2-dimensional surface may be selected from autologous tissue cells, homologous or heterologous tissue cells, autologous stem cells, and homologous or heterologous stem cells.

Autologous tissue cells may be selected from fibroblast, chondrocyte, osteoblast, endothelial cell, myoblast, smooth muscle cell, hepatocyte, neural cell, cardiomyocyte, and interverteveral disc cell.

Homologous or heterologous tissue cells may be selected from fibroblast, chondrocyte, osteoblast, endothelial cell, myoblast, smooth muscle cell, hepatocyte, neural cell, cardiomyocyte, and interverteveral disc cell.

Autologous stem cells may be selected from embryonic stem cells, dedifferentiation stem cells, bone marrow-derived stem cells, adipose-derived stem cells, placenta-derived stem cells, and myo-derived stem cells

Homologous or heterologous tissue cells may be selected from embryonic stem cells, dedifferentiation stem cells, bone marrow-derived stem cells, adipose-derived stem cells, placenta-derived stem cells, and myo-derived stem cells

In addition, the method may include decellularizing of the cells cultured on the 2-dimensional surface. The decellularizing is performed to remove cells from the extracellular matrix, and the decellularizing may be performed by a physical method, a chemical method, or a mixture thereof.

According to an example of a physical decellularization method, cells are frozen and then, are ruptured by iced crystals formed therein. Alternatively, a physical pressure may be applied to cells, so that the cells are damaged due to the physical force. Alternatively, cells are stirred (agitation or sonication) by using a magnetic bar to rupture and remove the cells.

According to an example of a chemical decellularization method, the cultured cells may be immersed in a decellularization solution. In detail, intracellular components are removed by using an acidic solution, such as an acetic acid, a peracetic acid (PAA), a hydrochloric acid, or a sulfuric acid. Alternatively, Triton X-100 as a non-ionic detergent is used to rupture cell membrane to remove intracellular components. Alternatively, DNase and RNase are used to remove cell nucleus components. As the ionic detergent, conventionally, sodium dodecyl sulfate (SDS), sodium deoxycholate, or Triton X-200 is used, and such ionic detergents are used to decompose a cell membrane and a nucleus membrane, so that a protein-protein reaction is removed, leading to the cell rupturing. Alternatively, cells may be contained in a storage solution and then added to a hypertonic solution to induce rupturing of cells due to osmotic phenomenon. Alternatively, ethylenediaminetetraacetic acid (EDTA) or ethylene glycol tetraacetic acid (EGTA) is used as a chelating agent to rupture and remove cells. As an enzymatic method, when trypsin is used together with EDTA at the temperature of 37° C. and at a pH of 8.0, cells are ruptured and intracellular components thereof are removed.

In addition, the method may further include obtaining an extracellular matrix from the 2-dimensional surface. The extracellular matrix may be obtained as a thin film.

According to an embodiment of the present invention, the method may further include adding a bioactive material to a medium to promote cell proliferation or secretion of extracellular matrix during culturing of cells on the 2-dimensional surface.

The bioactive material may be any one selected from the group consisting of transforming growth factor, fibroblast growth factor, bone morphogenetic protein, vascular endothelial growth factor, epidermal growth factor, insulin-like growth factor, platelet-derived growth factor, nerve growth factor, hepatocyte growth factor, placental growth factor, and granulocyte colony stimulating factor.

The present invention also provides a self-assembled matrix that consists of extracellular matrices.

The self-assembled matrix that consists of extracellular matrices is formed by culturing cells on a 2-dimensional surface to form an extracellular matrix, decellularizing the 2-dimensional surface on which cells are cultured, and obtaining the extracellular matrix.

The present invention also provides a method of inducing in vitro cell proliferation or differentiation, the method including seeding the self-assembled matrix that consists of extracellular matrices on cells and culturing the seeded cells.

The seeded cells may be animal cells.

The seeded cells may be tissue cells or stem cells.

The tissue cells may be autologous, homologous, or heterologous tissue cells, and may be any one selected from the group consisting of fibroblasts, chondrocytes, osteoblasts, endothelial cells, smooth muscle cells, hepatocytes, nerve cells, cardiomyocytes, and intervertebral disc cells.

The cell proliferation may be evaluated by any one selected from the group consisting of Brd U assay, MTT assay, and CCK-8 assay.

In detail, Brd U assay is a method of evaluating cell proliferation in viable tissues. Brd U may be inserted, instead of thymine, into DNA that is newly synthesized in proliferated cells. The extent of cell proliferation is evaluated based on a quantity of DNA that is replicated by using an antibody that is specifically reactive to Brd U.

According to MTT assay, reducing of yellow MTT tetrasolium that is an aqueous matrix into bluish violet non-aqueous MTT formazan due to dehydrogenases that is present in an electron delivery system of mitochondria of viable cells is evaluated by spectrophotometry at a wavelength of 540 nm and the number of viable cells is counted based on the results.

According to CCK-8 assay (cell counting kit-8), like MTT assay, an aqueous tetrasolium salt is decomposed into formazan due to dehydrogenases in an electron delivery system of mitochondria of viable cells and the number of viable cells is counted based on the results.

In addition, the cell differentiation is to differentiate stem cells into a particular cell, wherein stem cells may be selected from the group consisting of embryonic stem cells, dedifferentiation stem cells, bone marrow-derived stem cells, adipose-derived stem cells, placenta-derived stem cells, and myo-derived stem cells, and the particular cell may be selected from the group consisting of chondrocytes, osteoblasts, endothelial cells, endothelial precursor cells, myoblast cells, smooth muscle cells, hepatocytes, nerve cells, and cardiomyocytes.

In addition, the cell differentiation may be identified by detecting protein or mRNA existing in differentiated tissue cells. For example, the cell differentiation may be identified by tissue staining, safranin O staining, immunohistochemistry, or PCR.

The present invention also provides a method of evaluating cell proliferation or differentiation, the method including seeding a self-assembled matrix that consists of extracellular matrices on stem cells and in vitro evaluating proliferation or differentiation of the seeded stem cells.

The stem cells may be autologous, homologous, or heterologous stem cells.

The cell differentiation is to differentiate stem cells into a particular cell, wherein stem cells may be selected from the group consisting of embryonic stem cells, dedifferentiation stem cells, bone marrow-derived stem cells, adipose-derived stem cells, placenta-derived stem cells, and myo-derived stem cells, and the particular cell may be selected from the group consisting of chondrocytes, osteoblasts, endothelial cells, endothelial precursor cells, myoblast cells, smooth muscle cells, hepatocytes, nerve cells, cardiomyocytes.

In addition, the method may further include adding a material for promoting proliferation or differentiation of stem cells when differentiation on the self-assembled matrix is evaluated. The extracellular matrix is a material that is the most similar to a live body environment. Accordingly, the material for promoting proliferation or differentiation of stem cells may be a material for promoting proliferation or differentiation of stem cells in vivo.

Accordingly, a material for promoting proliferation or differentiation of stem cells may be screened by using such assays.

Another embodiment of the present invention provides a cell therapy product including as an active component cells that are proliferated or differentiated by culturing on a self-assembled matrix.

In this regard, the cell therapy product may include a self-assembled matrix. In addition, the cells may be autologous, homologous, or heterologous cells.

As an example of the cells, stem cells may be selected from the group consisting of embryonic stem cells, dedifferentiation stem cells, bone marrow-derived stem cells, adipose-derived stem cells, placenta-derived stem cells, and myo-derived stem cells, and when the cells are differentiated into a particular cell, the particular cell may be selected from the group consisting of chondrocytes, osteoblasts, endothelial cells, endothelial precursor cells, myoblast cells, smooth muscle cells, hepatocytes, nerve cells, cardiomyocytes.

As another example of the cells, tissue cells may be selected from the group consisting of chondrocytes, dedifferentiated chondrocytes, osteoblasts, endothelial cells, endothelial precursor cells, myoblast cells, smooth muscle cells, hepatocytes, nerve cells, and cardiomyocytes.

Another embodiment of the present invention provides a method of treating disease to a human individual having the disease by administrating the cell therapy product thereto.

The disease to which the cell therapy product is applicable may be selected from the group consisting of autoimmune disease, cardiovascular disease, bone disease, and nervous disease, but is not limited thereto.

Hereinafter, the present invention is described in detail with reference to examples and experimental examples. However, the present invention is not limited thereto.

### EXAMPLE 1

### Cell Growth on Cover Slip

To grow a particular cell, cells were inoculated with a density of 2×10⁴ cells/cm² on a cover slip for cell growth, and cells were cultured on a culture plate for a predetermined period of time while the medium was exchanged every two days. Examples of an adhesive cell include bone-derived stem cells and various primary cells and cell strains are used. The present experiments were performed by using fibroblasts, chondrocytes, and osteoblasts to form a self-assembled matrix.

### EXAMPLE 2

### Preparation of Self-Assembled Matrix

Five to six days after the culturing, confluent cells underwent decellularization to remove a nucleus of cells to obtain cell-derived extracellular matrix.

In detail, cells cultured on the cover slip were washed with PBS and then immersed in a decellularization solution. The decellularization solution mainly consisted of 0.25% Triton X-100, 10 mM NH₄OH, and a PBS solution, which were present in a mixed form. The sample was immersed in the decellularization solution at the temperature of 37° C. for 2 minutes, and then the solution was removed therefrom. Thereafter, the resulting sample was immersed in 50 unit/ml of DNase and 50 mg/ml of RNase solution and then treated in an incubator at the temperature of 37° C. for 2 hours.

The sample was taken out therefrom, and then, carefully washed with PBS, and the obtained matrix structure in a film shape was treated with a 0.1 M glycine-PBS solution and then preserved at the temperature of 4° C.

### EXAMPLE 3

### Self-Assembled Matrix Structure and Components Assay

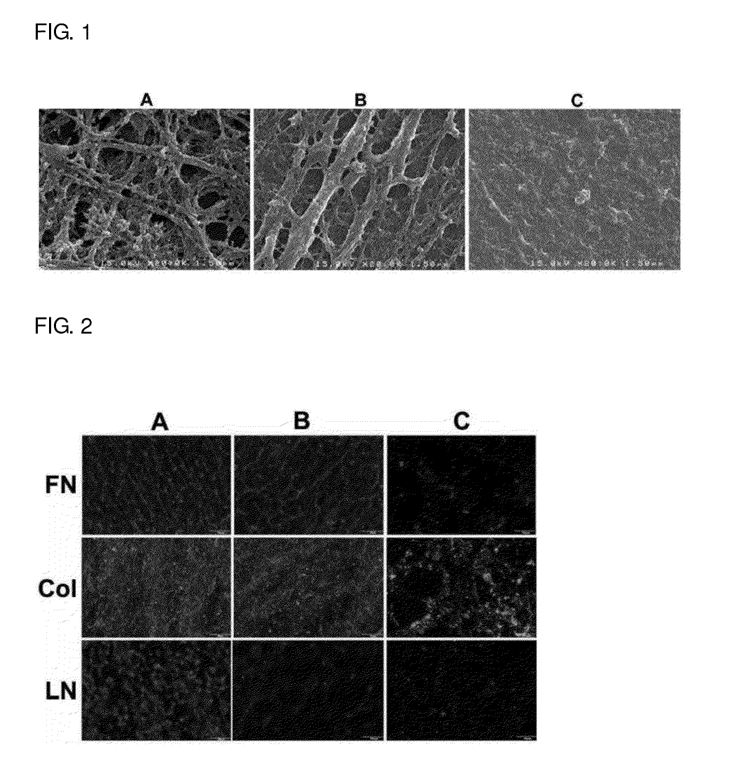

This method was performed to analyze components and surface structure of cell-derived extracellular matrix by using a confocal microscope (Olympus Fluo View™ FV1000) and a scanning electron microscope (scanning electron microscope) (SEM; HITACHI S-410, Tokyo, Japan). Nano structure assay of the extracellular matrix was performed as follows: the matrix prepared according to Example 2 was washed with PBS, and then, fixed at room temperature with 2.5% glutaraldehyde/2.5% paraformaldehyde for 15 minutes, and then, finally dehydrated with an ethanol/water mixed solution. Thereafter, the result was placed in a vacuum oven to be completely dried, and then, subjected to plutonium/palladium coating. The obtained scaffold was identified by FE-SEM (see FIG. 1).

To analyze constituting components of the self-assembled matrix, the sample was washed with PBS, and then, at room temperature, fixed with 4% paraformaldehyde for 15 minutes and then, fixed with cold acetone at the temperature of −20° C. for 10 minutes, and then, completely dried at room temperature. The dried sample was washed with PBS, and then, non-specific protein binding was suppressed with 3% bovine serum albumin (BSA) solution. In addition, the sample was treated with a primary antibody of corresponding component, fibronectin (rat monoclonal IgG), Col 1 (goat polyclonal IgG), and laminin (goat polyclonal IgG), which were diluted with a concentration of 1:50 in 1% BSA solution. The resulting sample was preserved at the temperature of 4° C. for 12 hours, and then, reacted at room temperature for about 1 hour with a 1:200 dilution of one of Alexa Flour 488-conjugated goat anti-rat IgG (Invitrogen) and DyLight 594-conjugated anti-goat IgG (Jackson Immuno Research), which are secondary antibodies to which Alexa Fluor 488 (Invitrogen, Eugene, Oreg.) or Alexa Fluor 594 (Jackson Immunoresearch, West Grove, Pa.) as a fluorescent material binds. The reaction product was washed with PBS. Then, an aqueous mounting medium (Permanent mounting medium, Vexta Mount™) was mounted on the sample, and then, a corresponding component (fibronectin, type one collagen, laminin) was analyzed under a confocal fluorescent microscope (FIG. 2).

### EXAMPLE 4

### Cell Adhesion and Proliferation on Self-Assembled Matrix

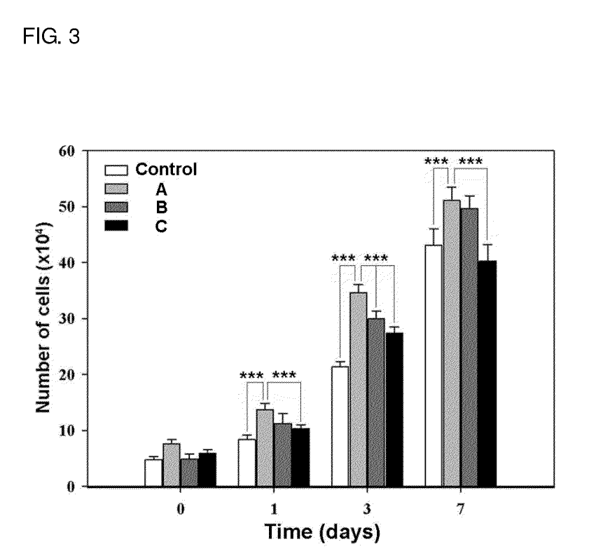

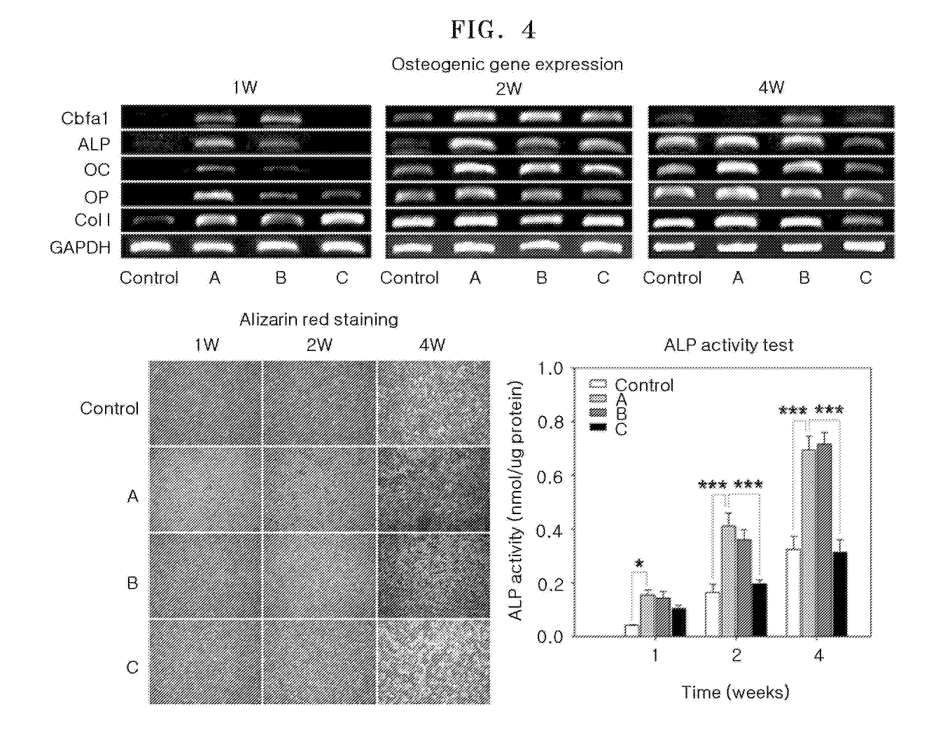

The present experiment was performed to evaluate cell adhesion and proliferation by seeding and culturing cells on the matrix prepared according to Example 2 at a concentration of 5×10³ cells/cm². The used matrices were fibroblast-derived matrix (FDM), preosteoblast-derived matrix (PDM), and chondrocyte-derived matrix (CHDM). The seeded cells were bone marrow stromal cells (BMSC). Stem cells were seeded on matrices obtained from different cells, and then, within 24 hours after the seeding, cell morphology was identified by fluorescent staining using F-actin and cell proliferation was evaluated using CCK-8 assay (Cell Counting Kit-8, Dojindo, Japan) by measuring the cell number change during 2 weeks. The number of cells continuously increased over time, and in particular, the cell proliferation was high on the matrices (see FIG. 3).

### EXAMPLE 5

### BMSC Differentiation Induction on Self-Assembled Matrix

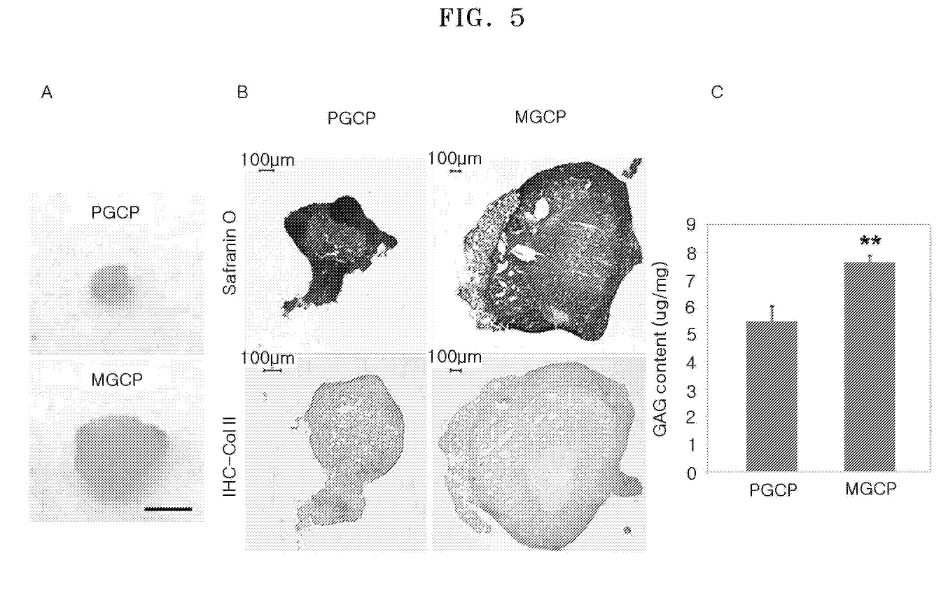

BMSC isolated from bone marrow of rat was seeded on the respective matrices, and incubated in an osteogenic medium for 4 weeks to induce osteogenic differentiation of BMSC. The present experiment was performed as follows: histological staining, ALP activity (alkaline phosphatase activity) measurement, and bone marker gene expression were performed, and then, results thereof were analyzed to evaluate osteogenic potentials of the respective matrices (see FIG. 4).

Bone specific histological staining (Alizarin red staining) results showed that bone differentiation of groups A (FDM) and B (PDM) progressed well, which was identified by staining concentration and distribution. Likewise, when ALP activity of cells was measured, groups A and B showed substantially high activities. In addition, in terms of bone specific gene expression, the bone specific gene expression of group A was relatively strongly maintained. Accordingly, it was confirmed that the self-assembled matrix positively affected bone differentiation of bone marrow stem cells.

### EXAMPLE 6

### Studies on Chondrocyte Mass Proliferation and Cartilage Differentiation on Self-Assembled Matrix

Preosteoblasts were seeded on a cell culturing plate and cultured for 6 days in a modified Eagle's medium alpha (α-MEM) with 10% fetal bovine serum and 1% penicillin/streptomycin, and then, subjected to decellularization, thereby forming a “PDM plate” on which matrices were adhered. Separately, chondrocytes were isolated from the knee articular cartilage of rats and sub-cultured to passage 3 using a typical cell plate to induce cell proliferation. The obtained chondrocytes were then seeded on two different plates (TCP and PDM plate). Thereafter, the cells were sub-cultured to passage 5 under the same culture conditions, and then, collected. A suspension containing 1×10⁶ chondrocytes was placed in 15 ml tube, and centrifuged for 10 minutes at 1200 rpm. Cell pellets formed on the bottom of the tube were identified, and were divided into two groups (TCP culturing vs. PDM plate culturing) and the groups were cultured under the same conditions for 4 weeks. Thereafter, for cartilage tissue assay, histological staining (safranin O staining), immuno histological staining (immunohistochemistry of type II collagen), quantitative glycosaminoglycan (GAG) assay were carried out in order to identify cartilage differentiation of proliferated chondrocyte on a matrix plate or a cell culture plate (see FIG. 5).

When a self-assembled matrix that consists of extracellular matrices according to the present invention is used, proliferation and differentiation of tissue cells or stem cells may be effectively induced. The proliferated and differentiated tissue cells or stem cells may be used in various cell therapy products. Accordingly, a method according to the present invention, a self-assembled matrix obtained by using the method, and a cell proliferation and differentiation method using the self-assembled matrix can be usefully utilized. Also, since the obtained cells are used in cell therapy product, the present invention is applicable in a variety of industrial use.

A matrix prepared by using a method of preparing a self-assembled matrix that consists of extracellular matrices derived from cells according to embodiments of the present invention may provide a microenvironment that is effective for proliferation and differentiation of cells. In particular, since on a matrix that consists of nanofiber, stem cells may retain its undifferentiation state for a long period of time or differentiation of the stem cells into a particular cell may be promoted, stem cell therapy products can be developed or important materials or information related to tissue regeneration may be provided.

Also, the self-assembled matrix that consists of extracellular matrices derived from cells according to the present invention may provide a platform that is required for cell proliferation and differentiation into a particular cell. Also, a great number of cells obtained by a plurality of sub-culturing on the self-assembled matrix may be used alone or in combination with the matrix for (matrix-assisted) cell therapy.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of preparing a self-assembled matrix that consists of extracellular matrices, the method comprising:
culturing cells on a 2-dimensional surface to form extracellular matrices;
decellularizing the cells cultured on the 2-dimensional surface; and
obtaining an extracellular matrix from the 2-dimensional surface.

2. The method of claim 1, wherein the cells are tissue cells or stem cells.

3. The method of claim 2, wherein the tissue cells are selected from fibroblasts, chondrocytes, osteoblasts, endothelial cells, myoblasts, smooth muscle cells, hepatocytes, neural cells, cardiomyocytes, and interverteveral disc cells.

4. The method of claim 2, wherein the stem cells are selected from the group consisting of embryonic stem cells, dedifferentiation stem cells, bone marrow-derived stem cells, adipose-derived stem cells, placenta-derived stem cells, and myo-derived stem cells.

5. The method of claim 1, further comprising adding a bioactive material to a medium to proliferate cells or to promote secretion of extracellular matrix during cells are cultured on the 2-dimensional surface.

6. The method of claim 5, wherein the bioactive material is any one selected from the group consisting of transforming growth factor, fibroblast growth factor, bone morphogenetic protein, vascular endothelial growth factor, epidermal growth factor, insulin-like growth factor, platelet-derived growth factor, nerve growth factor, hepatocyte growth factor, placental growth factor, and granulocyte colony stimulating factor.

7. A self-assembled matrix that consists of extracellular matrices for cell adhesion and the promotion of proliferation.

8. The self-assembled matrix of claim 7, wherein the self-assembled matrix that consists of extracellular matrices is prepared by using the method of claim 1.

9. A method of inducing cell proliferation or differentiation in vitro, the method comprising:
1) seeding cells on the self-assembled matrix of claim 7; and
2) culturing the seeded cells

10. The method of claim 9, wherein the seeded cells are tissue cells or stem cells.

11. The method of claim 10, wherein the tissue cells are autologous, homologous, or heterologous tissue cells.

12. The method of claim 15, wherein the tissue cells are selected from fibroblasts, chondrocytes, osteoblasts, endothelial cells, smooth muscle cells, hepatocytes, neural cells, cardiomyocytes, and interverteveral disc cells.

13. The method of claim 10, wherein the stem cells are selected from the group consisting of embryonic stem cells, dedifferentiation stem cells, bone marrow-derived stem cells, adipose-derived stem cells, placenta-derived stem cells, and myo-derived stem cells.

14. A cell therapy product comprising cells that proliferated or differentiated on a self-assembled matrix by using the method of claim 9.

15. The cell therapy product of claim 14, wherein the proliferated or differentiated cells are tissue cells or stem cells.

16. A method for the treatment of disease, the method comprising administering proliferated or differentiated cells on a self-assembled matrix by using the method of claim 9 to an individual having a disease.

17. The method of claim 16, wherein the disease is any one selected from the group consisting of autoimmune disease, cardiovascular disease, bone disease, and nervous disease.
